(19) United States
(12) Patent Application Publication
(10) Pub. No.: US 2009/0247553 A1
(43) Pub. Date: Oct. 1, 2009

(54) Highly Pure Paliperidone or a Pharmaceutically Acceptable Salt Thereof Substantially Free of Keto Impurity

(75) Inventors: Uday Rajaram Bapat, Mumbai (IN); Munusamy Jayamani, Chennai (IN); Sivaji Ravisaravanan, Madurai (IN); Vishal Amrutlal Sodha, Gujarat (IN); Jon Valgeirsson, Hafnarfjordur (IS)

Correspondence Address: CANTOR COLBURN, LLP, 20 Church Street, 22nd Floor, Hartford, CT, 06103, US

(73) Assignee: ACTAVIS GROUP PTC EHF, Hafnarfjordur (IS)

(21) Appl. No.: 12/411,526

(22) Filed: Mar. 26, 2009

(30) Foreign Application Priority Data
Mar. 27, 2008 (IN) 748/CHE/2008
Jun. 13, 2008 (IN) 1442/CHE/2008

(51) Int. Cl. A61K 31/519; C07D 239/70; A61P 25/18

(52) U.S. Cl. 514/259.41; 544/282

(57) ABSTRACT

Provided herein is a highly pure paliperidone or a pharmaceutically acceptable salt thereof substantially free of keto impurity, 3-[2-[4-(6-fluoro-1,2-benzisoxazol-3-yl)-1-piperidinyl]ethyl]-2-methyl-7,8-dihydro-6H-pyrido[1,2-a]pyrimidin-4,9-dione, a process for the preparation thereof, and pharmaceutical compositions comprising highly pure paliperidone or a pharmaceutically acceptable salt thereof substantially free of keto impurity.

### CROSS REFERENCE TO RELATED APPLICATION

This application claims the benefit of priority to Indian provisional application Nos. 748/CHE/2008, filed on March 27, 2008; and 1442/CHE/2008, filed on Jun. 13, 2008; which are incorporated herein by reference in their entirety.

### FIELD OF THE DISCLOSURE

Disclosed herein is a highly pure paliperidone or a pharmaceutically acceptable salt thereof substantially free of keto impurity, 3-[2-[4-(6-fluoro-1,2-benzisoxazol-3-yl)-1-piperidinyl]ethyl]-2-methyl-7,8-dihydro-6H-pyrido[1,2-a]pyrimidin-4,9-dione, a process for the preparation thereof, and pharmaceutical compositions comprising highly pure paliperidone or a pharmaceutically acceptable salt thereof substantially free of keto impurity.

### BACKGROUND

U.S. Pat. Nos. 4,804,663 and 5,158,952 disclose a variety of 3-piperidinyl-1,2-benzisoxazole derivatives, processes for their preparation, pharmaceutical compositions comprising the derivatives, and methods of use thereof. These compounds have long-acting antipsychotic properties and are useful in the treatment of warm-blooded animals suffering from psychotic diseases. Among them, paliperidone, (±)-3-[2-[4-(6-fluoro-1,2-benzisoxazol-3-yl)-1-piperidinyl]ethyl]-6,7,8,9-tetrahydro-9-hydroxy-2-methyl-4H-pyrido[1,2-a]pyrimidin-4-one, is an antipsychotic agent and indicated for the both acute (short-term) and maintenance (long-term) treatment of schizophrenia. Paliperidone is represented by the following structural formula:

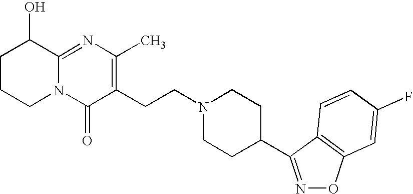

Processes for the preparation of paliperidone and related compounds are disclosed in U.S. Pat. Nos. 5,158,952; 5,254,556; 5,688,799 and 6,320,048.

According to U.S. Pat. No. 5,158,952 (hereinafter referred to as the '952 patent), paliperidone is prepared by the reaction of 3-(2-chloroethyl)-6,7,8,9-tetrahydro-9-hydroxy-2-methyl-4H-pyrido[1,2-a]-pyrimidin-4-one with 6-fluoro-3-(4-piperidinyl)-1,2-benzisoxazole in the presence of a base in a reaction inert solvent and optionally in the presence of a phase transfer catalyst.

The reaction inert solvents include water; an aromatic solvent, e.g., benzene, methylbenzene, dimethylbenzene, chlorobenzene, methoxybenzene and the like; a C₁₋₆ alkanol, e.g. methanol, ethanol, 1-butanol and the like; a ketone, e.g., 2-propanone, 4-methyl-2-pentanone and the like; an ester, e.g. ethyl acetate, γ-butyrolactone and the like; an ether, e.g. 1,1′-oxybisethane, tetrahydrofuran, 1,4-dioxane and the like; a dipolar aprotic solvent, e.g. N,N-dimethylformamide, N,N-dimethylacetamide, dimethylsulfoxide, pyridine, 1,3-dimethyl-3,4,5,6-tetrahydro-2(1H)-pyrimidinone, 1,3-dimethyl-2-imidazolidinone, 1,1,3,3-tetramethylurea, 1-methyl-2-pyrrolidinone, nitrobenzene, acetonitrile and the like; or a mixture thereof. The bases include inorganic bases such as, for example, an alkali metal or an earth alkaline metal carbonate, hydrogen carbonate, hydroxide, oxide, carboxylate, alkoxide, hydride or amide, e.g., sodium carbonate, sodium hydrogen carbonate, potassium carbonate, sodium hydroxide, calcium oxide, sodium acetate, sodium methoxide, sodium hydride, sodium amide and the like, or an organic base such as, for example, a tertiary amine, e.g. N,N-diethylethanamine, N-(1-methylethyl)-2-propanamine, 4-ethylmorpholine, 1,4-diazabicyclo[2.2.2]octane, pyridine and the like. The phase transfer catalysts include trialkylphenylmethylammonium, tetraalkylammonium, tetraalkylphosphonium, tetraarylphosphonium halide, hydroxide, hydrogen sulfate, and the like.

The reaction mixture containing paliperidone obtained according to the method of the '952 patent is then subjected to evaporation and the oily residue is extracted with trichloromethane followed by water washings. The organic layer is dried, filtered and evaporated followed by column chromatographic purifications over silica gel using a mixture of trichloromethane and methanol. The pure fractions are collected and the eluent is evaporated. The resulting residue is crystallized from 2-propanone. After cooling, the precipitated product is filtered off, washed with a mixture of 2-propanol and 2,2′-oxybispropane, and recrystallized from 2-propanol to produce paliperidone.

Paliperidone obtained by the process described in the '952 patent does not have satisfactory purity for pharmaceutical use. Unacceptable amounts of impurities are generally formed along with paliperidone. In addition, the process involves the additional step of column chromatographic purifications. Methods involving column chromatographic purifications are generally undesirable for large-scale operations, thereby making the process commercially unfeasible.

It is known that synthetic compounds can contain extraneous compounds or impurities resulting from their synthesis or degradation. They can be unreacted starting materials, by-products of the reaction, products of side reactions, or degradation products. Generally, impurities in an active pharmaceutical ingredient (API) may arise from degradation of the API itself, or during the preparation of the API. Impurities in paliperidone or any active pharmaceutical ingredient (API) are undesirable and might be harmful.

Regulatory authorities worldwide require that drug manufactures isolate, identify and characterize the impurities in their products. Furthermore, it is required to control the levels of these impurities in the final drug compound obtained by the manufacturing process and to ensure that the impurity is present in the lowest possible levels, even if structural determination is not possible.

The product mixture of a chemical reaction is rarely a single compound with sufficient purity to comply with pharmaceutical standards. Side products and byproducts of the reaction and adjunct reagents used in the reaction will, in most cases, also be present in the product mixture. At certain stages during processing of the active pharmaceutical ingredient, the product must be analyzed for purity, typically, by HPLC, TLC or GC analysis, to determine if it is suitable for continued processing and, ultimately, for use in a pharmaceutical product. Purity standards are set with the intention of ensuring that an API is as free of impurities as possible, and, thus, are as safe as possible for clinical use. The United States Food and Drug Administration guidelines recommend that the amounts of some impurities limited to less than 0.1 percent.

Generally, impurities are identified spectroscopically and by other physical methods, and then the impurities are associated with a peak position in a chromatogram (or a spot on a TLC plate). Thereafter, the impurity can be identified by its position in the chromatogram, which is conventionally measured in minutes between injection of the sample on the column and elution of the particular component through the detector, known as the “retention time” (“Rt”). This time period varies daily based upon the condition of the instrumentation and many other factors. To mitigate the effect that such variations have upon accurate identification of an impurity, practitioners use “relative retention time” (“RRt”) to identify impurities. The RRt of an impurity is its retention time divided by the retention time of a reference marker.

It is known by those skilled in the art, the management of process impurities is greatly enhanced by understanding their chemical structures and synthetic pathways, and by identifying the parameters that influence the amount of impurities in the final product.

PCT Publication No. 2008/021346 (herein after referred to as the '346 application) teaches four general purification methods of paliperidone. According to the first purification process, paliperidone is crystallized from a solvent selected from the group consisting of: C₃₋₆ ketone or a mixture thereof with water, N-methylpyrrolidone, C₃₋₆ amides, halo-substituted C₆₋₁₂ aromatic hydrocarbons, propylene glycol, dimethyl sulfoxide, di-methyl carbonate, C₁₋₄ alkyl alcohols, a mixture of a C₁₋₈ alkyl alcohol and water, acetonitrile or a mixture thereof with water, C₂₋₆ alkyl acetates or their mixture with water, cellosolve, dimethyl carbonate, polyethylene glycol methyl ether and C₂₋₈ ethers. The crystallization involves dissolving paliperidone in the above solvents, heating the reaction mixture to allow complete dissolution, followed by cooling of the obtained solution, whereby paliperidone crystallizes. The second purification process of the '346 application comprises crystallizing paliperidone by combining a solution of paliperidone in a first solvent, selected from the group consisting of: dichloromethane, dioxane and C₁₋₄ alkyl alcohols, with an anti-solvent selected from the group consisting of C₃₋₆ ketones, C₃₋₆ ethers, acetonitrile, C₃₋₇ straight and cyclic carbohydrates, C₆₋₁₂ aromatic carbohydrates and water. The third purification process of the '346 application comprises slurrying paliperidone in an organic solvent selected from C₁₋₄ alkyl alcohols, C₃₋₅ ketones and water. The fourth purification process of the '346 application comprises dissolving paliperidone in a mixture of acetone and water, admixing the solution with finely powdered carbon, and filtrating the admixture to obtain pure paliperidone.

There is a need for pure paliperidone or a pharmaceutically acceptable salt thereof substantially free of keto impurity with reduced particle size distribution, which has good flow properties, and better dissolution and solubility properties to obtain formulations with greater bioavailability.

### SUMMARY

In one aspect, provided herein is a highly pure paliperidone or a pharmaceutically acceptable salt thereof substantially free of paliperidone keto impurity, 3-[2-[4-(6-fluoro-1,2-benzisoxazol-3-yl)-1-piperidinyl]ethyl]-2-methyl-7,8-dihydro-6H-pyrido[1,2-a]pyrimidin-4,9-dione.

As used herein, “highly pure paliperidone or a pharmaceutically acceptable salt thereof substantially free of paliperidone keto impurity” refers to paliperidone or a pharmaceutically acceptable salt thereof, in which paliperidone has a purity of about 99% to about 99.99% and further comprising paliperidone keto impurity in an amount of less than about 0.2 wt % as measured by HPLC. Specifically, the paliperidone, as disclosed herein, contains less than about 0.15 wt %, more specifically less than about 0.05 wt %, still more specifically less than about 0.02 wt % of paliperidone keto impurity, and most specifically is essentially free of paliperidone keto impurity.

In another aspect, provided herein is paliperidone or a pharmaceutically acceptable salt thereof that comprises paliperidone keto impurity in an amount of about 0.01 wt % to about 0.15 wt %, specifically in an amount of about 0.01 wt % to about 0.05 wt %, as measured by HPLC.

In another aspect, provided herein is paliperidone or a pharmaceutically acceptable salt thereof having a total purity of greater than about 99%, specifically greater than about 99.5%, more specifically greater than about 99.9%, and most specifically greater than about 99.95% as measured by HPLC.

In still further aspect, encompassed herein is a process for preparing the highly pure paliperidone or a pharmaceutically acceptable salt thereof substantially free of paliperidone keto impurity.

Preferable pharmaceutically acceptable salts of paliperidone include, but are not limited to, hydrochloride, hydrobromide, oxalate, nitrate, sulphate, phosphate, fumarate, succinate, maleate, fumarate, besylate, tosylate, tartrate, palmitate; and more preferably hydrochloride.

In another aspect, provided herein is a pharmaceutical composition comprising highly pure paliperidone or a pharmaceutically acceptable salt thereof substantially free of paliperidone keto impurity, and one or more pharmaceutically acceptable excipients.

In still another aspect, provided herein is a pharmaceutical composition comprising highly pure paliperidone or a pharmaceutically acceptable salt thereof substantially free of paliperidone keto impurity made by the process disclosed herein, and one or more pharmaceutically acceptable excipients.

In still further aspect, encompassed is a process for preparing a pharmaceutical formulation comprising combining highly pure paliperidone or a pharmaceutically acceptable salt thereof substantially free of paliperidone keto impurity with one or more pharmaceutically acceptable excipients.

In another aspect, the highly pure paliperidone or a pharmaceutically acceptable salt thereof substantially free of paliperidone keto impurity disclosed herein for use in the pharmaceutical compositions has a 90 volume-percent of the particles (D₉₀) having a size of less than or equal to about 400 microns, specifically less than or equal to about 300 microns, more specifically less than or equal to about 100 microns, still more specifically less than or equal to about 60 microns, and most specifically less than or equal to about 15 microns.

### DETAILED DESCRIPTION

Extensive experimentation has been carried out by the present inventors to reduce the level of the keto impurity in paliperidone. As a result, it has been found that the keto impurity formed in the preparation of the paliperidone can be reduced by treating crude paliperidone with a reducing agent in a suitable solvent, and then isolating substantially pure paliperidone.

Paliperidone obtained by the processes described in the art is not satisfactory from a purity perspective. Reproduction of the paliperidone synthetic procedures as described in the prior art show that unacceptable amounts of impurities are generally formed along with paliperidone. Among these impurities, 3-[2-[4-(6-fluoro-1,2-benzisoxazol-3-yl)-1-piperidinyl]ethyl]-2-methyl-7,8-dihydro-6H-pyrido[1,2-a]pyrimidin-4,9-dione (hereinafter referred to as the ‘keto impurity’) of formula I:

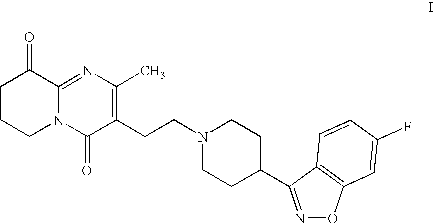

is the main concern, and it is identified and isolated. The structure of the compound of formula I was deduced with the aid of ¹H, ¹³C NMR spectroscopy and FAB mass spectroscopy. The parent ion at 424.4 is consistent with assigned structure. In a specific experiment, the inventors have found that paliperidone prepared by the above prior art procedures contained about above 1 wt % and up to 3 wt % of the keto impurity. The present inventors conducted experiments to purify the crude paliperidone and found that the content of the keto impurity could be further reduced to about 0.5 wt % by using the above mentioned re-crystallization procedures described in the prior art. However, the amount of keto impurity could not be reduced to below 0.2 wt % or eliminated completely using the prior art purification procedures.

Accordingly, there remains a need for highly pure paliperidone substantially free of the keto impurity, as well as purification processes for obtaining thereof.

In addition, the solid state physical properties of an active pharmaceutical ingredient (API), such as paliperidone, can be very important in formulating a drug substance and can have profound effects on the ease and reproducibility of formulation. Particle size, for example, may affect the flowability and mixability of a drug substance. In cases where the active ingredient has good flow properties, tablets can be prepared by direct compression of the ingredients. However, in many cases, the particle size of the active substance is very small, the active substance is cohesive, or the active substance has poor flow properties. Small particles are also filtered and washed more slowly during isolation processes, and thus may increase the time and expense of manufacturing a drug formulation.

Paliperidone is a white to yellow non-hygroscopic powder and poorly soluble in water. Its solubility in water is 0.003 g/100 ml, increasing to 2.3 g/100 ml in 0.1N HCl. In ethanol the solubility of paliperidone is 0.076 g/100 ml. The poor solubility of paliperidone is problematic since bioavailability of a water insoluble active ingredient is usually poor. There is a need to prepare active pharmaceutical ingredients such as paliperidone particles with a desired surface area to obtain formulations with greater bioavailability, and to compensate for any loss of surface area before formulation.

According to one aspect, provided herein is a highly pure paliperidone or a pharmaceutically acceptable salt thereof substantially free of paliperidone keto impurity, 3-[2-[4-(6-fluoro-1,2-benzisoxazol-3-yl)-1-piperidinyl]ethyl]-2-methyl-7,8-dihydro-6H-pyrido[1,2-a]pyrimidin-4,9-dione.

As used herein, “highly pure paliperidone or a pharmaceutically acceptable salt thereof substantially free of paliperidone keto impurity” refers to paliperidone or a pharmaceutically acceptable salt thereof, in which paliperidone has a purity of about 99% to about 99.99% and further comprising a paliperidone keto impurity in an amount of less than about 0.2 wt % as measured by HPLC. For example, the purity of the highly pure paliperidone is about 99% to about 99.95%, or about 99.5% to about 99.99%. Specifically, the paliperidone, as disclosed herein, contains less than about 0.15 wt %, more specifically less than about 0.05 wt %, still more specifically less than about 0.02 wt % of paliperidone keto impurity, and most specifically is essentially free of paliperidone keto impurity.

In one embodiment, the highly pure paliperidone or a pharmaceutically acceptable salt thereof disclosed herein comprises a paliperidone keto impurity in an amount of about 0.01 wt % to about 0.15 wt %, specifically in an amount of about 0.01 wt % to about 0.05 wt %, as measured by HPLC.

The term “paliperidone or a pharmaceutically acceptable salt thereof essentially free of paliperidone keto impurity” refers to paliperidone or a pharmaceutically acceptable salt thereof contains a non-detectable amount of paliperidone keto impurity as measured by HPLC.

Exemplary pharmaceutically acceptable salts of paliperidone include, but are not limited to, hydrochloride, hydrobromide, oxalate, nitrate, sulfate, phosphate, fumarate, succinate, maleate, fumarate, besylate, tosylate, tartrate, palmitate; and more specifically hydrochloride.

According to another aspect, there is provided a process for the preparation of highly pure paliperidone or a pharmaceutically acceptable salt thereof substantially free of keto impurity, comprising:

a) providing a first solution or a suspension of crude paliperidone in a first organic solvent;

b) isolating or recovering the paliperidone as a solid from the solution or suspension from step-(a);

c) dissolving the solid from (b) in a second organic solvent to form a second solution;

d) combining the second solution obtained in step-(c) with a reducing agent to produce a reaction mass;

e) optionally, filtering the reaction mass obtained in step-(d) to remove extraneous matter; and

isolating highly pure paliperidone substantially free of keto impurity from the reaction mass, and optionally converting the highly pure paliperidone obtained into a pharmaceutically acceptable salt.

In one embodiment, the process disclosed herein or any one of the process steps can be repeated any number of times until the substantial removal of the keto impurity and to provide the desired purity.

The first organic solvent used in step-(a) is selected from the group consisting of alcohols, amides, organosulfur solvents, and mixtures thereof Exemplary alcohol solvents include, but are not limited to, C₁ to C₆ straight or branched chain alcohol solvents such as methanol, ethanol, n-propanol, isopropanol, isobutanol, n-butanol, tert-butanol, amyl alcohol, isoamyl alcohol, hexanol, and mixtures thereof. Specific alcohol solvents are methanol, ethanol, isopropanol, and mixtures thereof, and most specific alcohol solvent is methanol. Exemplary amide solvents include, but are not limited to, C₃ to C₆ amides such as dimethylacetamide, dimethylformamide, and mixtures thereof. Specific amide solvent is dimethylformamide. Exemplary organosulfur solvents include, but are not limited to, thioethers, thioesters, thioacetals, thiols, sulfolane, dimethylsulfoxide, and mixtures thereof. A specific organosulfur solvent is sulfolane.

Specifically, the organic solvent is selected from the group consisting of methanol, ethanol, n-propanol, isopropanol, isobutanol, n-butanol, tert-butanol, amyl alcohol, isoamyl alcohol, dimethylacetamide, dimethylformamide, dimethylsulfoxide, sulfolane, and mixtures thereof; more specifically methanol, isopropanol, dimethylformamide, sulfolane, and mixtures thereof; and most specifically sulfolane.

Step-(a) of providing a solution of crude paliperidone includes dissolving crude paliperidone in the first organic solvent, or obtaining an existing solution from a previous processing step.

In one embodiment, the crude paliperidone is dissolved in the first organic solvent at a temperature of above about 25° C., specifically at about 25° C. to about 110° C., and more specifically at about 30° C. to about 80° C.

In another embodiment, step-(a) of providing a suspension of crude paliperidone includes suspending crude paliperidone in the first organic solvent while stirring at a temperature below boiling temperature of the solvent used. In one embodiment, the suspension is stirred at a temperature of about 15° C. to about 110° C. for at least 30 minutes, and more specifically at about 25° C. to about 80° C. from about 1 hour to about 10 hours.

In another embodiment, the solution or suspension in step-(a) is prepared by reacting 3-(2-chloroethyl)-6,7,8,9-tetrahydro-9-hydroxy-2-methyl-4H-pyrido[1,2-a]-pyrimidin-4-one with 6-fluoro-3-(4-piperidinyl)-1,2-benzisoxazole in the presence of a base, optionally in the presence of a phase transfer catalyst, in a reaction inert solvent under suitable conditions to produce a reaction mass containing crude paliperidone followed by usual work up such as washings, extractions, evaporations etc., and dissolving or suspending the resulting crude paliperidone in the first organic solvent at a temperature of above about 25° C., specifically at about 25° C. to about 110° C., and more specifically at about 30° C. to about 80° C.

Exemplary phase transfer catalysts suitable for facilitating the reaction between 3-(2-chloroethyl)-6,7,8,9-tetrahydro-9-hydroxy-2-methyl-4H-pyrido[1,2-a]-pyrimidin-4-one and 6-fluoro-3-(4-piperidinyl)-1,2-benzisoxazole include, but are not limited to, quaternary ammonium salts substituted with a group such as a straight or branched alkyl group having 1 to about 18 carbon atoms, a phenyl lower alkyl group including a straight or branched alkyl group having 1 to 6 carbon atoms which is substituted by an aryl group and phenyl group, e.g., tetrabutylammonium chloride, tetrabutylammonium bromide, tetrabutylammonium fluoride, tetrabutylammonium iodide, tetrabutylammonium hydroxide, tetrabutylammonium hydrogen sulfate, tributylmethylammonium chloride, tributylbenzylammonium chloride, tetraethylammonium chloride, tetramethylammonium chloride, tetrapentylammonium chloride, tetrapentylammonium bromide, tetrahexyl ammonium chloride, benzyldimethyloctylammonium chloride, methyltrihexylammonium chloride, benzylmethyloctadecanylammonium chloride, methyltridecanylammonium chloride, benzyltripropylammonium chloride, benzyltriethyl ammonium chloride, phenyltriethylammonium chloride and the like; phosphonium salts substituted with a residue such as a straight or branched alkyl group having 1 to about 18 carbon atoms, e.g., tetrabutylphosphonium chloride and the like; and pyridinium salts substituted with a straight or branched alkyl group having 1 to about 18 carbon atoms, e.g., 1-dodecanylpyridinium chloride and the like.

Specific phase transfer catalysts are tetrabutylammonium bromide, tetrabutylphosphonium bromide, tetrabutylammonium chloride, tetrabutylphosphonium chloride, benzyltriethylammonium chloride, tetrabutylammonium hydrogen sulfate, and more specifically tetrabutylammonium bromide.

Exemplary reaction inert solvents suitable for facilitating the reaction between 3-(2-chloroethyl)-6,7,8,9-tetrahydro-9-hydroxy-2-methyl-4H-pyrido[1,2-a]-pyrimidin-4-one and 6-fluoro-3-(4-piperidinyl)-1,2-benzisoxazole include, but are not limited to, water, alcohols, ketones, cyclic ethers, aliphatic ethers, hydrocarbons, chlorinated hydrocarbons, nitriles, esters, polar aprotic solvents, and the like, and mixtures thereof. In one embodiment, the solvent is selected from the group consisting of water, methanol, ethanol, n-propanol, isopropanol, n-butanol, isobutanol, tert-butanol, amyl alcohol, hexanol, acetone, methyl ethyl ketone, methyl isobutyl ketone, methyl tert-butyl ketone, acetonitrile, ethyl acetate, methyl acetate, isopropyl acetate, tert-butyl methyl acetate, ethyl formate, dichloromethane, dichloroethane, chloroform, carbon tetrachloride, tetrahydrofuran, dioxane, diethyl ether, diisopropyl ether, monoglyme, diglyme, n-pentane, n-hexane, n-heptane, cyclohexane, toluene, xylene, N,N-dimethylformamide, N,N-dimethylacetamide, dimethylsulfoxide, and mixtures thereof.

In one embodiment, the base suitable for facilitating the reaction between 3-(2-chloroethyl)-6,7,8,9-tetrahydro-9-hydroxy-2-methyl-4H-pyrido[1,2-a]-pyrimidin-4-one and 6-fluoro-3-(4-piperidinyl)-1,2-benzisoxazole is an organic or inorganic base. Specific organic bases are triethyl amine, dimethyl amine and tert-butyl amine.

In another embodiment, the base is an inorganic base. Exemplary inorganic bases include, but are not limited to, aqueous ammonia; hydroxides, carbonates and bicarbonates of alkali or alkaline earth metals. Specific inorganic bases are aqueous ammonia, sodium hydroxide, calcium hydroxide, magnesium hydroxide, potassium hydroxide, lithium hydroxide, sodium carbonate, potassium carbonate, lithium carbonate, sodium tert-butoxide, sodium isopropoxide and potassium tert-butoxide, and more specifically sodium hydroxide, potassium hydroxide, sodium carbonate and potassium carbonate.

Alternatively, the solution or suspension in step-(a) is prepared by treating an acid addition salt of paliperidone with a base to liberate paliperidone followed by extracting, dissolving or suspending the paliperidone in the first organic solvent at a temperature of above about 25° C., specifically at about 25° C. to about 110° C., and more specifically at about 30° C. to about 80° C.

In another embodiment, the acid addition salt of paliperidone is derived from a therapeutically acceptable acid such as hydrochloric acid, hydrobromic acid, acetic acid, propionic acid, sulfuric acid, nitric acid, phosphoric acid, succinic acid, maleic acid, fumaric acid, citric acid, glutaric acid, citraconic acid, glutaconic acid, tartaric acid, malic acid, and ascorbic acid. A specific salt is paliperidone hydrochloride.

The treatment of an acid addition salt with a base is carried out in a solvent and the selection of solvent is not critical. A wide variety of solvents such as chlorinated solvents, alcohols, ketones, hydrocarbon solvents, esters, ether solvents etc., can be used.

The base used herein can be selected from inorganic and organic bases as described above.

The solution or suspension obtained in step-(a) is optionally stirred at a temperature of about 30° C. to the reflux temperature of the solvent used for at least 20 minutes, and specifically at a temperature of about 40° C. to the reflux temperature of the solvent used from about 30 minutes to about 6 hours.

The isolation of paliperidone in step-(b) is carried out, for example, by forcible or spontaneous crystallization.

Spontaneous crystallization refers to crystallization without the help of an external aid, such as seeding, cooling etc., and forcible crystallization refers to crystallization with the help of an external aid.

Forcible crystallization is initiated by methods such as cooling, seeding, partial removal of the solvent from the solution, by combining an anti-solvent with the solution, or a combination thereof.

In one embodiment, the crystallization is carried out by cooling the solution at a temperature of below 30° C. for at least 30 minutes, specifically at about 0° C. to about 25° C. from about 1 hour to about 20 hours, and more specifically at about 0° C. to about 20° C. from about 2 hours to about 10 hours.

The recovery of solid paliperidone in step-(b) is accomplished by techniques such as filtration, filtration under vacuum, decantation, centrifugation, or a combination thereof. In one embodiment, the solid paliperidone is recovered by filtration employing a filtration media of, for example, a silica gel or celite.

In one embodiment, the solid paliperidone obtained in step-(b) is optionally washed with solvents such as water, alcohols, and mixtures thereof prior to dissolving in the second organic solvent.

The second organic solvent used in step-(c) is selected from the group consisting of C₁ to C₆ straight or branched chain alcohol solvents such as methanol, ethanol, n-propanol, isopropanol, isobutanol, n-butanol, tert-butanol, amyl alcohol, isoamyl alcohol, hexanol, and mixtures thereof. Specific alcohol solvents are methanol, ethanol, isopropanol, and mixtures thereof, and most specifically methanol.

In one embodiment, the dissolution in step-(c) is carried out at a temperature of above about 25° C., specifically at about 25° C. to about 110° C., and more specifically at about 40° C. to about 80° C.

The solution obtained in step-(c) is optionally subjected to carbon treatment or silica gel treatment. The carbon treatment or silica gel treatment is carried out by methods known in the art, for example, by stirring the solution with finely powdered carbon or silica gel at a temperature of below about 70° C. for at least 15 minutes, specifically at a temperature of about 40° C. to about 70° C. for at least 30 minutes; and filtering the resulting mixture through hyflo to obtain a filtrate containing paliperidone by removing charcoal or silica gel. Preferably, finely powdered carbon is an active carbon. A specific mesh size of silica gel is 40-500 mesh, and more specifically 60-120 mesh.

In another embodiment, the solution obtained in step-(c) is optionally stirred at a temperature of about 30° C. to the reflux temperature of the solvent used for at least 10 minutes, and specifically at a temperature of about 40° C. to the reflux temperature of the solvent used from about 15 minutes to about 5 hours.

The reducing agent used in step-(d) includes metal hydrides such as sodium borohydride, sodium cyanoborohydride, sodium bis(2-methoxyethoxy)aluminium hydride, lithium borohydride, potassium borohydride, and combinations comprising one or more of the foregoing reducing agents. A specific reducing agent is sodium borohydride.

Combining of the solution with reducing agent in step-(d) is done in a suitable order, for example, the solution is added to the reducing agent, or alternatively, the reducing agent is added to the solution. The addition is, for example, carried out drop wise or in one portion or in more than one portion. The addition is specifically carried out at a temperature of below about 60° C., and more specifically at a temperature of about 25° C. to about 55° C. After completion of addition process, the resulting mass is stirred at a temperature of about 20° C. to about 70° C., and more specifically at about 35° C. to about 55° C. Specifically, the stirring is performed for a period of time sufficient for purifying paliperidone, more specifically at least for about 10 minutes and still more specifically from about 30 minutes to about 10 hours.

In one embodiment, the reducing agent used in step-(d) in the molar ratio of about 0.002 to 0.15 moles, specifically 0.005 to 0.01 moles, per 1 mole of paliperidone in order to ensure reduction or complete removal of the keto impurity.

The isolation of highly pure paliperidone substantially free of keto impurity in step-(f) is carried out by forcible or spontaneous crystallization.

In one embodiment, the crystallization is carried out by cooling the solution at a temperature of below 30° C. for at least 30 minutes, specifically at about 0° C. to about 25° C. from about 30 minutes to about 20 hours, and more specifically at about 0° C. to about 20° C. from about 1 hour to about 8 hours.

The solid obtained in step-(f) is collected by filtration, filtration under vacuum, decantation, centrifugation, or a combination thereof In one embodiment, the highly pure paliperidone is collected by filtration employing a filtration media of, for example, a silica gel or celite.

The highly pure paliperidone obtained by the above process may be further dried in, for example, a Vacuum Tray Dryer, Rotocon Vacuum Dryer, Vacuum Paddle Dryer or pilot plant Rota vapor, to further lower residual solvents. Drying can be carried out under reduced pressure until the residual solvent content reduces to the desired amount such as an amount that is within the limits given by the International Conference on Harmonization of Technical Requirements for Registration of Pharmaceuticals for Human Use (“ICH”) guidelines.

In one embodiment, the drying is carried out at atmospheric pressure or reduced pressures, such as below about 200 mm Hg, or below about 50 mm Hg, at temperatures such as about 35° C. to about 70° C. The drying can be carried out for any desired time period that achieves the desired result, such as times about 1 to 20 hours. Drying may also be carried out for shorter or longer periods of time depending on the product specifications. Temperatures and pressures will be chosen based on the volatility of the solvent being used and the foregoing should be considered as only a general guidance. Drying can be suitably carried out in a tray dryer, vacuum oven, air oven, or using a fluidized bed drier, spin flash dryer, flash dryer, and the like. Drying equipment selection is well within the ordinary skill in the art.

Paliperidone obtained by the process disclosed herein specifically contains the paliperidone keto impurity in an amount of less than about 0.2 wt %, more specifically less than about 0.05 wt %, still more specifically less than about 0.02 wt %, and most specifically essentially free of paliperidone keto impurity as measured by HPLC.

The purity of the paliperidone obtained after the purification process disclosed herein is of greater than about 99%, specifically greater than about 99.5%, more specifically greater than about 99.9%, and most specifically greater than about 99.95% as measured by HPLC. For example, the purity of the paliperidone of the present invention is about 99% to about 99.95%, or about 99.5% to about 99.99%.

In another embodiment, the highly pure paliperidone substantially free of keto impurity obtained in step-(f) is optionally converted into pharmaceutically acceptable salts by conventional methods.

According to another aspect, there is provided a process for purifying paliperidone, comprising:

a) providing a first solution of paliperidone in an organic solvent;

b) subjecting the first solution to silica gel treatment to produce a second solution; and

c) isolating highly pure paliperidone from the second solution obtained in step-(b), and optionally converting the highly pure paliperidone obtained into its pharmaceutically acceptable salts.

The organic solvent used in step-(a) is selected from the group consisting of alcohols, amides, organosulfur solvents, and mixtures thereof Specifically, the organic solvent is selected from the group consisting of methanol, ethanol, n-propanol, isopropanol, isobutanol, n-butanol, tert-butanol, amyl alcohol, isoamyl alcohol, dimethylacetamide, dimethylformamide, dimethylsulfoxide, sulfolane, and mixtures thereof; and more specifically methanol, isopropanol, dimethylformamide, sulfolane, and mixtures thereof.

In an embodiment, the solution in step-(a) is provided by the methods disclosed herein above.

The silica gel treatment in step-(b) is carried out by stirring the solution with silica gel at a temperature of below about 70° C. for at least 10 minutes, specifically at a temperature of about 40° C. to about 70° C. from about 15 minutes to about 5 hours; and filtering the resulting mixture through hyflo to obtain a filtrate containing pure paliperidone by removing silica gel. A specific mesh size of silica gel is 40-500 mesh, and more specifically 60-120 mesh.

The isolation of highly pure paliperidone in step-(c) is carried out by forcible or spontaneous crystallization methods.

Pharmaceutically acceptable salts of paliperidone can be prepared in high purity by using the highly pure paliperidone substantially free of keto impurity obtained by the method disclosed herein, by known methods, for example as described in U.S. Pat. No. 4,804,663.

Specific pharmaceutically acceptable salts of paliperidone include, but are not limited to, hydrochloride, hydrobromide, oxalate, nitrate, sulphate, phosphate, fumarate, succinate, maleate, fumarate, besylate, tosylate, palmitate and tartrate; and more specifically hydrochloride.

The term “crude paliperidone” refers to paliperidone containing the keto impurity in an amount of greater than 0.2 wt %.

Further encompassed herein is the use of the highly pure paliperidone or a pharmaceutically acceptable salt thereof substantially free of keto impurity for the manufacture of a pharmaceutical composition together with a pharmaceutically acceptable carrier.

A specific pharmaceutical composition of highly pure paliperidone or a pharmaceutically acceptable salt thereof substantially free of keto impurity is selected from a solid dosage form and an oral suspension.

In one embodiment, the highly pure paliperidone or a pharmaceutically acceptable salt thereof substantially free of keto impurity has a D₉₀ particle size of less than or equal to about 400 microns, specifically less than or equal to about 300 microns, more specifically less than or equal to about 100 microns, still more specifically less than or equal to about 60 microns, and most specifically less than or equal to about 15 microns.

In another embodiment, the particle sizes of the highly pure paliperidone or a pharmaceutically acceptable salt thereof substantially free of keto impurity are produced by a mechanical process of reducing the size of particles which includes any one or more of cutting, chipping, crushing, milling, grinding, micronizing, trituration or other particle size reduction methods known in the art, to bring the solid state form to the desired particle size range.

According to another aspect, there is provided a method for treating a patient suffering from psychotic diseases, comprising administering a therapeutically effective amount of the highly pure paliperidone or a pharmaceutically acceptable salt thereof substantially free of keto impurity, or a pharmaceutical composition that comprises a therapeutically effective amount of highly pure paliperidone or a pharmaceutically acceptable salt thereof substantially free of keto impurity, along with pharmaceutically acceptable excipients.

According to another aspect, there is provided pharmaceutical compositions comprising highly pure paliperidone or a pharmaceutically acceptable salt thereof substantially free of keto impurity prepared according to processes disclosed herein and one or more pharmaceutically acceptable excipients.

According to another aspect, there is provided a process for preparing a pharmaceutical formulation comprising combining highly pure paliperidone or a pharmaceutically acceptable salt thereof substantially free of keto impurity prepared according to processes disclosed herein, with one or more pharmaceutically acceptable excipients.

Yet in another embodiment, pharmaceutical compositions comprise at least a therapeutically effective amount of highly pure paliperidone or a pharmaceutically acceptable salt thereof substantially free of keto impurity. Such pharmaceutical compositions may be administered to a mammalian patient in a dosage form, e.g., solid, liquid, powder, elixir, aerosol, syrups, injectable solution, etc. Dosage forms may be adapted for administration to the patient by oral, buccal, parenteral, ophthalmic, rectal and transdermal routes or any other acceptable route of administration. Oral dosage forms include, but are not limited to, tablets, pills, capsules, syrup, troches, sachets, suspensions, powders, lozenges, elixirs and the like. The highly pure paliperidone or a pharmaceutically acceptable salt thereof substantially free of keto impurity may also be administered as suppositories, ophthalmic ointments and suspensions, and parenteral suspensions, which are administered by other routes.

The dosage forms may contain highly pure paliperidone or a pharmaceutically acceptable salt thereof substantially free of keto impurity as is or, alternatively, may contain highly pure paliperidone or a pharmaceutically acceptable salt thereof substantially free of keto impurity as part of a composition. The pharmaceutical compositions may further contain one or more pharmaceutically acceptable excipients. Suitable excipients and the amounts to use may be readily determined by the formulation scientist based upon experience and consideration of standard procedures and reference works in the field, e.g., the buffering agents, sweetening agents, binders, diluents, fillers, lubricants, wetting agents and disintegrants described hereinabove.

In one embodiment, capsule dosage forms contain highly pure paliperidone or a pharmaceutically acceptable salt thereof substantially free of keto impurity within a capsule which may be coated with gelatin. Tablets and powders may also be coated with an enteric coating. The enteric-coated powder forms may have coatings containing at least phthalic acid cellulose acetate, hydroxypropylmethyl cellulose phthalate, polyvinyl alcohol phthalate, carboxy methyl ethyl cellulose, a copolymer of styrene and maleic acid, a copolymer of methacrylic acid and methyl methacrylate, and like materials, and if desired, the coating agents may be employed with suitable plasticizers and/or extending agents. A coated capsule or tablet may have a coating on the surface thereof or may be a capsule or tablet comprising a powder or granules with an enteric-coating.

Tableting compositions may have few or many components depending upon the tableting method used, the release rate desired and other factors. For example, the compositions described herein may contain diluents such as cellulose-derived materials like powdered cellulose, microcrystalline cellulose, microfine cellulose, methyl cellulose, ethyl cellulose, hydroxyethyl cellulose, hydroxypropyl cellulose, hydroxypropylmethyl cellulose, carboxymethyl cellulose salts and other substituted and unsubstituted celluloses; starch; pregelatinized starch; inorganic diluents such calcium carbonate and calcium diphosphate and other diluents known to one of ordinary skill in the art. Yet other suitable diluents include waxes, sugars (e.g. lactose) and sugar alcohols such as mannitol and sorbitol, acrylate polymers and copolymers, as well as pectin, dextrin and gelatin.

Other excipients include binders, such as acacia gum, pregelatinized starch, sodium alginate, glucose and other binders used in wet and dry granulation and direct compression tableting processes; disintegrants such as sodium starch glycolate, crospovidone, low-substituted hydroxypropyl cellulose and others; lubricants like magnesium and calcium stearate and sodium stearyl fumarate; flavorings; sweeteners; preservatives; pharmaceutically acceptable dyes and glidants such as silicon dioxide.

The highly pure paliperidone can be used in the treatment of psychotic diseases such as schizophrenia.

The following examples are given for the purpose of illustrating the present disclosure and should not be considered as limitation on the scope or spirit of the disclosure.

### Experimental:

### High Performance Liquid Chromatography (HPLC):

The HPLC purity was measured by high performance liquid chromatography by using Waters, alliance 2695 HPLC system having dual wavelength UV detector under the following conditions:

| | |
|---|---|
| Column: | ACE-3 C18 |
| | (15 cm × 4.6 mm × 3 um) |
| Column oven temperature: | 30° C. |
| Detection: | 237 nm |
| Flow rate: | 1 ml/min |
| Injection volume: | 20 μl |
| Run time: | 35 min |
| Diluent: | MeOH:ACN:water 1:1:1 |

### EXAMPLES

### Example 1

### Step-I: Preparation of Crude Paliperidone

The mixture of 3-(2-chloroethyl)-6,7,8,9-tetrahydro-9-hydroxy-2-methyl-4H-pyrido[1,2-a]-pyrimidin-4-one (60 g), 6-fluoro-3-(4-piperidinyl)-1,2-benzisoxazole (48.4 g), sodium carbonate (91.87 g) and ethanol (1200 ml) was stirred for 24 hours at 60-65° C. under a nitrogen atmosphere. After completion of the reaction, the reaction mass was filtered and then ethanol was distilled off completely. The product was extracted with methylene chloride (1000 ml) and the methylene chloride layer was washed with water and then dried over sodium sulphate. Methylene chloride was distilled out completely, the product was diluted with methanol (200 ml), and then stirred for 2-3 hours at 30-35° C. The solid obtained was isolated by filtration, washed with methanol (50 ml) and then dried under vacuum at 40-45° C. to produce 50 g of crude paliperidone (Content of the keto impurity: 0.53 wt % as measured by HPLC).

### Step-II: Purification of Paliperidone

Crude paliperidone (50 g, obtained in step-I) was stirred with dimethylformamide (250 ml) at 50-60° C. for 3 hours and the resulting mass was cooled to 25° C. The product was then filtered followed by washings with dimethylformamide (50 ml) and methanol (50 ml). The wet solid was stirred with dimethylformamide (150 ml) for 3 hours at 50-60° C., the resulting mass was cooled to 25° C. and then filtered. The filtered solid was washed with dimethylformamide (50 ml) followed by methanol (50 ml). The resulting wet product was stirred with methanol (250 ml) for 3 hours at 30° C., and the solid was filtered and washed with methanol to produce paliperidone (Wet weight: 28 g). The product was then added to methanol (1800 ml) and the resulting mixture was heated to 65° C. under stirring to get a clear solution. The solution was cooled to 50° C. followed by addition of activated carbon (7.5 g) and then the mixture was stirred for 30 minutes at 50° C. The resulting hot solution was filtered through hyflo to remove charcoal. Sodium borohydride (25 mg) was added into filtrate and stirred for 2 hours at 50° C. The solution was then concentrated to one-fourth volume under vacuum and then cooled to 25° C. The separated solid was filtered, washed with methanol and then dried to produce 24 g of pure paliperidone (HPLC Purity: 99.85%; Content of the keto impurity: 0.05 wt %).

### Example 2

### Step-I: Preparation of Crude Paliperidone

3-(2-Chloroethyl)-6,7,8,9-tetrahydro-9-hydroxy-2-methyl-4H-pyrido[1,2-a]-pyrimidin-4-one (25 g) was added to ethanol (500 ml) at 25-30° C. under stirring. 6-Fluoro-3-(4-piperidinyl)-1,2-benzisoxazole (20.1 g) and anhydrous sodium carbonate (38 g) were added to the above solution and then heated to 58-62° C. The resulting mass was stirred for 24 hours at 58-62° C. The reaction mass was cooled to 25° C., the inorganic material was filtered out and the resulting mass was washed with ethanol (75 ml). The ethanol was distilled under vacuum in water bath at 50° C. and the resulting residue was dissolved in methylene dichloride (1250 ml). The methylene chloride solution was washed with water (600 ml) 3 times and then dried with anhydrous sodium sulfate. Sodium sulfate was filtered and methylene dichloride was distilled under vacuum at 30-40° C. After complete distillation of methylene chloride, methanol (75 ml) was added to the residue, stirred for 2 hours, filtered the material and dried at 50° C. under high vacuum to yield 23.0 g of crude paliperidone (HPLC Purity: 95.11%; content of the keto impurity: 0.24 wt %).

### Step-II: Purification of Paliperidone

Crude paliperidone (23 g, obtained in step-I) was heated with dimethylformamide (115 ml) under stirring at 56° C. for 3 hours. The resulting mass was cooled to 25° C., and material was filtered and washed initially with dimethylformamide (23 ml) followed by methanol (23 ml). The resulting solid was heated to 56° C. with dimethylformamide (69 ml) and the mass was stirred for 3 hours and then cooled to 25° C. The resulting mass was filtered and washed initially with dimethylformamide (23 ml) followed by methanol (23 ml). The resulting solid was stirred with methanol (80 ml) for 3 hours, filtered, washed with methanol (80 ml), and then dried in an air oven at 25° C. for 3 hours to yield 13.6 g of paliperidone. The product was then dissolved in methanol (816 ml) at 65° C. to provide a clear solution and the solution was cooled to 50° C. This procedure was followed by the addition of activated carbon (3.5 gm). The resulting mass was stirred for 30 minutes at 50° C. and then filtered on a Hiflo bed. Sodium borohydride (10 mg) was added to the resulting solution and then stirred for 2 hours at 50° C. followed by distillation of methanol until the paliperidone crystallized out. The resulting solution was cooled to 25° C., filtered the material, washed with methanol and then dried at 60° C. under vacuum to yield 10.7 g of pure paliperidone (HPLC Purity: 99.95%; Content of the keto impurity: 0.035 wt %).

### Example 3

### Step-I: Preparation of Paliperidone (Crude)

3-(2-Chloroethyl)-6,7,8,9-tetrahydro-9-hydroxy-2-methyl-4H-pyrido[1,2-a]-pyrimidin-4-one (25 g) was added to ethanol (500 ml) at 25-30° C. under stirring. This addition was followed by the addition of6-fluoro-3-(4-piperidinyl)-1,2-benzisoxazole (20.1 g) and diisopropylethyl amine (24.8 g). The resulting mass was heated to 58-62° C. and then stirred for 24 hours at 58-62° C. The resulting mass was cooled to 25° C. followed by removal of ethanol under vacuum at 50° C. and the resulting residue was dissolved in methylene chloride (1250 ml). The methylene chloride solution was washed with water (600 ml) 3 times and then dried with anhydrous sodium sulfate. Sodium sulfate was filtered and methylene chloride was distilled under vacuum at 30-40° C. After complete distillation of methylene chloride, methanol (75 ml) was added to the residue with stirring for 2 hours. The material was filtered and dried at 50° C. under high vacuum to yield 14.0 g of crude paliperidone (HPLC Purity: 96.12%; Content of the keto impurity: 0.68 wt % at 0.96 RRt).

### Step-II: Purification of Paliperidone

Crude paliperidone (10 g, obtained in step-I) was heated with sulfolane (50 ml) under stirring at 60° C. for 3 hours, the resulting mass was cooled to 25° C. The material was filtered and washed initially with sulfolane (10 ml) followed by methanol (10 ml). Sulfolane (30 ml) was added to the above solid, the resulting mixture was heated to 60° C. under stirring and then maintained for 3 hours. The resulting mass was cooled to 25° C., and the solid was filtered and washed initially with sulfolane (10 ml) followed by methanol (10 ml). The filtered solid was stirred with water (125 ml) for 1 hour. The material was filtered and washed with water (50 ml) followed by methanol (25 ml). The resulting solid was then stirred with methanol (50 ml) for 1 hour, and the solid material was filtered and washed with methanol (25 ml) and then dried in an air oven at 25° C. for 3 hours to yield 5.3 g of paliperidone. The product was then dissolved in methanol (320 ml) at 65° C. to provide a clear solution. The solution was cooled to 50° C. followed by the addition of silica gel (10 g) and then stirred for 30 minutes at 50° C. The resulting solution was filtered to remove the silica gel. Sodium borohydride (10 mg) was added to the resulting filtrate, and the temperature was maintained for 2 hours at 50° C. Methanol was distilled until paliperidone crystallized out and the resulting solution was cooled to 25° C. The separated solid was filtered, washed with methanol and then dried at 60° C. under vacuum to yield 3.1 g of pure paliperidone (HPLC Purity 99.73%; Content of the keto impurity at 0.96 RRT: below detectable limit).

### Example 4

### Purification of Paliperidone

Crude paliperidone (20 g, obtained in step-I of example 2) was heated with sulfolane (100 ml) under stirring at 60° C. for 3 hours, the resulting mass was cooled to 25° C. The material was filtered and washed initially with sulfolane (20 ml) followed by methanol (20 ml). The filtered solid was slurried with sulfolane (60 ml), the resulting slurry was heated to 60° C. under stirring and then maintained for 3 hours. The resulting mass was cooled to 25° C., filtered the solid and washed initially with sulfolane (20 ml) followed by methanol (20 ml). The filtered solid was stirred with water (200 ml) for 1 hour and then the material was filtered and washed with water (100 ml) followed by methanol (50 ml). The resulting solid was then stirred with methanol (100 ml) for 1 hour, and the material was filtered and washed with methanol (50 ml) and then dried in an air oven at 25° C. for 3 hours to yield 13.3 g of paliperidone. The product was then dissolved in methanol (800 ml) at 65° C. to provide a clear solution. The solution was cooled to 50° C. followed by the addition of silica gel (20 g) with stirring for 30 minutes at 50° C. The resulting solution was filtered to remove the silica gel. Sodium borohydride (10 mg) was added to the resulting filtrate and the temperature was maintained for 2 hours at 50° C. Methanol was distilled until paliperidone crystallized out and the resulting solution was cooled to 25° C. The separated solid was filtered, washed with methanol and then dried at 60° C. under vacuum to yield 11 g of pure paliperidone (HPLC Purity 99.7%; Content of the keto impurity at 0.96 RRT: 0.02 wt %).

Unless otherwise indicated, the following definitions are set forth to illustrate and define the meaning and scope of the various terms used to describe the invention herein.

The term “paliperidone” as used herein refers to a racemic mixture of enantiomeric forms of paliperidone or an enatiomerically enriched form of paliperidone.

The term “pharmaceutically acceptable” means that which is useful in preparing a pharmaceutical composition that is generally non-toxic and is not biologically undesirable and includes that which is acceptable for veterinary use and/or human pharmaceutical use.

The term “pharmaceutical composition” is intended to encompass a drug product including the active ingredient(s), pharmaceutically acceptable excipients that make up the carrier, as well as any product which results, directly or indirectly, from combination, complexation or aggregation of any two or more of the ingredients. Accordingly, the pharmaceutical compositions encompass any composition made by admixing the active ingredient, active ingredient dispersion or composite, additional active ingredient(s), and pharmaceutically acceptable excipients.

The term “therapeutically effective amount” as used herein means the amount of a compound that, when administered to a mammal for treating a state, disorder or condition, is sufficient to effect such treatment. The “therapeutically effective amount” will vary depending on the compound, the disease and its severity and the age, weight, physical condition and responsiveness of the mammal to be treated.

The term “delivering” as used herein means providing a therapeutically effective amount of an active ingredient to a particular location within a host causing a therapeutically effective blood concentration of the active ingredient at the particular location. This can be accomplished, e.g., by topical, local or by systemic administration of the active ingredient to the host.

The term “buffering agent” as used herein is intended to mean a compound used to resist a change in pH upon dilution or addition of acid of alkali. Such compounds include, by way of example and without limitation, potassium metaphosphate, potassium phosphate, monobasic sodium acetate and sodium citrate anhydrous and dehydrate and other such material known to those of ordinary skill in the art.

The term “sweetening agent” as used herein is intended to mean a compound used to impart sweetness to a formulation. Such compounds include, by way of example and without limitation, aspartame, dextrose, glycerin, mannitol, saccharin sodium, sorbitol, sucrose, fructose and other such materials known to those of ordinary skill in the art.

The term “binders” as used herein is intended to mean substances used to cause adhesion of powder particles in granulations. Such compounds include, by way of example and without limitation, acacia, alginic acid, tragacanth, carboxymethylcellulose sodium, polyvinylpyrrolidone, compressible sugar (e.g., NuTab), ethylcellulose, gelatin, liquid glucose, methylcellulose, pregelatinized starch, starch, polyethylene glycol, guar gum, polysaccharide, bentonites, sugars, invert sugars, poloxamers (PLURONIC(™) F68, PLURONIC(™) F127), collagen, albumin, celluloses in non-aqueous solvents, polypropylene glycol, polyoxyethylene-polypropylene copolymer, polyethylene ester, polyethylene sorbitan ester, polyethylene oxide, microcrystalline cellulose, combinations thereof and other material known to those of ordinary skill in the art.

The term “diluent” or “filler” as used herein is intended to mean inert substances used as fillers to create the desired bulk, flow properties, and compression characteristics in the preparation of solid dosage formulations. Such compounds include, by way of example and without limitation, dibasic calcium phosphate, kaolin, sucrose, mannitol, microcrystalline cellulose, powdered cellulose, precipitated calcium carbonate, sorbitol, starch, combinations thereof and other such materials known to those of ordinary skill in the art.

The term “glidant” as used herein is intended to mean agents used in solid dosage formulations to improve flow-properties during tablet compression and to produce an anti-caking effect. Such compounds include, by way of example and without limitation, colloidal silica, calcium silicate, magnesium silicate, silicon hydrogel, cornstarch, talc, combinations thereof and other such materials known to those of ordinary skill in the art.

The term “lubricant” as used herein is intended to mean substances used in solid dosage formulations to reduce friction during compression of the solid dosage. Such compounds include, by way of example and without limitation, calcium stearate, magnesium stearate, mineral oil, stearic acid, zinc stearate, combinations thereof and other such materials known to those of ordinary skill in the art.

The term “disintegrant” as used herein is intended to mean a compound used in solid dosage formulations to promote the disruption of the solid mass into smaller particles which are more readily dispersed or dissolved. Exemplary disintegrants include, by way of example and without limitation, starches such as corn starch, potato starch, pregelatinized, sweeteners, clays, such as bentonite, microcrystalline cellulose (e.g., Avicel(™)), carsium (e.g., Amberlite(™)), alginates, sodium starch glycolate, gums such as agar, guar, locust bean, karaya, pectin, tragacanth, combinations thereof and other such materials known to those of ordinary skill in the art.

The term “wetting agent” as used herein is intended to mean a compound used to aid in attaining intimate contact between solid particles and liquids. Exemplary wetting agents include, by way of example and without limitation, gelatin, casein, lecithin (phosphatides), gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers (e.g., macrogol ethers such as cetomacrogol 1000), polyoxyethylene castor oil derivatives, polyoxyethylene sorbitan fatty acid esters, (e.g., TWEEN(™)s), polyethylene glycols, polyoxyethylene stearates colloidal silicon dioxide, phosphates, sodium dodecylsulfate, carboxymethylcellulose calcium, carboxymethylcellulose sodium, methylcellulose, hydroxyethylcellulose, hydroxyl propylcellulose, hydroxypropylmethylcellulose phthalate, noncrystalline cellulose, magnesium aluminum silicate, triethanolamine, polyvinyl alcohol, and polyvinylpyrrolidone (PVP). Tyloxapol (a nonionic liquid polymer of the alkyl aryl polyether alcohol type) is another useful wetting agent, combinations thereof and other such materials known to those of ordinary skill in the art.

As used herein, D_{X} means that X percent of the particles have a diameter less than a specified diameter D. Thus, a D₉₀ or d(0.9) of less than 300 microns means that 90 volume-percent of the particles in a composition have a diameter less than 300 microns.

The term “micronization” used herein means a process or method by which the size of a population of particles is reduced.

As used herein, the term “micron” or “μm” both are same refers to “micrometer” which is 1×10⁻⁶ meter.

As used herein, “crystalline particles” means any combination of single crystals, aggregates and agglomerates.

As used herein, “Particle Size Distribution (P.S.D)” means the cumulative volume size distribution of equivalent spherical diameters as determined by laser diffraction in Malvern Master Sizer 2000 equipment or its equivalent.

As used herein, the term, “detectable” refers to a measurable quantity measured using an HPLC method having a detection limit of 0.01 area-%.

As used herein, in connection with amount of impurities in paliperidone or a pharmaceutically acceptable salt thereof, the term “not detectable” means not detected by the herein described HPLC method having a detection limit for impurities of 0.01 area-%.

As used herein, “limit of detection (LOD)” refers to the lowest concentration of analyte that can be clearly detected above the base line signal, is estimated is three times the signal to noise ratio.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. The term wt % refers to percent by weight. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. Paliperidone or a pharmaceutically acceptable salt thereof comprising a paliperidone keto impurity in an amount of less than 0.2 wt % as measured by HPLC, wherein the paliperidone keto impurity is 3-[2-[4-(6-fluoro-1,2-benzisoxazol-3-yl)-1-piperidinyl]ethyl]-2-methyl-7,8-dihydro-6H-pyrido[1,2-a]pyrimidin-4,9-dione.

2. Paliperidone of claim 1, having a purity of about 99% to about 99.99% as measured by HPLC.

3. (canceled)

4. (canceled)

5. Paliperidone of claim 1, comprising the paliperidone keto impurity in an amount of about 0.01 wt % to about 0.15 wt %.

6. (canceled)

7. Paliperidone of claim 1, wherein the pharmaceutically acceptable salt of paliperidone is selected from the group consisting of hydrochloride, hydrobromide, oxalate, nitrate, sulfate, phosphate, fumarate, succinate, maleate, fumarate, besylate, tosylate, palmitate and tartrate.

8. (canceled)

9. A purification process for obtaining highly pure paliperidone or a pharmaceutically acceptable salt thereof having less than 0.2 wt % of a paliperidone keto impurity, comprising:
a) providing a first solution or suspension of crude paliperidone in a first organic solvent;
b) isolating or recovering the paliperidone as a solid from the first solution or suspension obtained in step-(a);
c) dissolving the solid from step-(b) in a second organic solvent to form a second solution;
d) combining the second solution obtained in step-(c) with a reducing agent to produce a reaction mass;
e) optionally, filtering the reaction mass obtained in step-(d) to remove extraneous matter; and
f) isolating highly pure paliperidone from the reaction mass, and optionally converting the highly pure paliperidone obtained into its pharmaceutically acceptable salts;
wherein the paliperidone keto impurity is 3-[2-[4-(6-fluoro-1,2-benzisoxazol-3-yl)-1-piperidinyl]ethyl]-2-methyl-7,8-dihydro-6H-pyrido[1,2-a]pyrimidin-4,9-dione.

10. The process of claim 9, wherein the first organic solvent used in step-(a) is selected from the group consisting of alcohols, amides, organosulfur solvents, and mixtures thereof.

11. The process of claim 10, wherein the organic solvent is selected from the group consisting of methanol, ethanol, n-propanol, isopropanol, isobutanol, n-butanol, tert-butanol, amyl alcohol, isoamyl alcohol, dimethylacetamide, dimethylformamide, dimethylsulfoxide, sulfolane, and mixtures thereof.

12. (canceled)

13. The process of claim 11, wherein the organic solvent is sulfolane.

14. The process of claim 9, wherein the first solution in step-(a) is provided by dissolving the crude paliperidone in the first organic solvent at a temperature of about 25° C. to about 110° C.

15. (canceled)

16. The process of claim 9, wherein the suspension in step-(a) is provided by suspending crude paliperidone in the first organic solvent while stirring at a temperature below boiling temperature of the solvent used.

17. (canceled)

18. (canceled)

19. The process of claim 9, wherein the first solution or suspension in step-(a) is prepared by reacting 3-(2-chloroethyl)-6,7,8,9-tetrahydro-9-hydroxy-2-methyl-4H-pyrido[1,2-a]-pyrimidin-4-one with 6-fluoro-3-(4-piperidinyl)-1,2-benzisoxazole in the presence of a base, optionally in the presence of a phase transfer catalyst, in a reaction inert solvent to produce a reaction mass containing crude paliperidone; subjecting the reaction mass to washings, extractions or evaporations; and dissolving or suspending the resulting crude paliperidone in the first organic solvent at a temperature of about 25° C. to about 110° C.

20. The process of claim 9, wherein the first solution or suspension in step-(a) is prepared by treating an acid addition salt of paliperidone with a base to produce paliperidone; and extracting, dissolving or suspending the paliperidone in first the organic solvent.

21. The process of claim 9, wherein the first solution or suspension obtained in step-(a) is optionally stirred at a temperature of about 30° C. to the reflux temperature of the solvent used from about 20 minutes to about 6 hours.

22. The process of claim 9, wherein isolating in step-(b) is carried out by forcible or spontaneous crystallization, and wherein the forcible crystallization is initiated by cooling, seeding, partial removal of the solvent from the solution, by adding an anti-solvent to the solution or a combination thereof.

23. (canceled)

24. (canceled)

25. The process of claim 22, wherein the crystallization is carried out by cooling the solution at about 0° C. to about 25° C. from about 30 minutes to about 20 hours.

26. The process of claim 9, wherein recovering of solid paliperidone in step-(b) is accomplished by filtration, filtration under vacuum, decantation, centrifugation, filtration employing a filtration media of a silica gel or celite, or a combination thereof.

27. (canceled)

28. The process of claim 9, wherein the solid paliperidone obtained in step-(b) is optionally washed with a solvent selected from the group consisting of water, alcohols, and mixtures thereof prior to dissolving in the second organic solvent.

29. The process of claim 9, wherein the second organic solvent used in step-(c) is selected from the group consisting of methanol, ethanol, n-propanol, isopropanol, isobutanol, n-butanol, tert-butanol, amyl alcohol, isoamyl alcohol, hexanol, and mixtures thereof.

30. (canceled)

31. The process of claim 29, wherein the solvent is methanol.

32. The process of claim 9, wherein the dissolution in step-(c) is carried out at a temperature of about 25° C. to about 110° C., and wherein the second solution obtained is optionally stirred at a temperature of about 30° C. to the reflux temperature of the solvent used for at least 10 minutes.

33. The process of claim 9, wherein the second solution obtained in step-(c) is optionally subjected to carbon treatment or silica gel treatment.

34. (canceled)

35. The process of claim 9, wherein the reducing agent used in step-(d) is selected from the group consisting of sodium borohydride, sodium cyanoborohydride, sodium bis(2-methoxyethoxy)aluminium hydride, lithium borohydride, potassium borohydride, and combinations comprising one or more of the foregoing reducing agents.

36. The process of claim 35, wherein the reducing agent is sodium borohydride.

37. The process of claim 9, wherein the reaction mass obtained in step-(d) is stirred at a temperature of about 20° C. to about 70° C. for at least about 10 minutes.

38. The process of claim 37, wherein the reaction mass is stirred at a temperature of about 35° C. to about 55° C. from about 30 minutes to about 10 hours.

39. The process of claim 9, wherein the reducing agent in step-(d) is used in a molar ratio of about 0.002 to 0.15 moles per 1 mole of paliperidone.

40. The process of claim 39, wherein the reducing agent is used in the molar ratio of about 0.005 to 0.01 moles per 1 mole of paliperidone.

41. The process of claim 9, wherein isolating in step-(f) is carried out by forcible or spontaneous crystallization, and wherein the forcible crystallization is initiated by cooling, seeding, partial removal of the solvent from the solution, by adding an anti-solvent to the solution, or a combination thereof.

42. (canceled)

43. (canceled)

44. The process of claim 41, wherein the crystallization is carried out by cooling the solution at about 0° C. to about 30° C. from about 30 minutes to about 20 hours.

45. The process of claim 9, wherein the solid obtained in step-(f) is collected by filtration, filtration under vacuum, decantation, centrifugation, filtration employing a filtration media of a silica gel or celite, or a combination thereof; and wherein the highly pure paliperidone obtained is further dried under vacuum or at atmospheric pressure, at a temperature of about 35° C. to about 70° C.

46. (canceled)

47. The process of claim 9, wherein the paliperidone obtained in step-(f) has a purity of about 99% to about 99.95%.

48. The process of claim 47, wherein the paliperidone contains the keto impurity in an amount of about 0.01 wt % to about 0.15 wt %.

49. The process of claim 47, wherein the paliperidone is essentially free of paliperidone keto impurity.

50. (canceled)

51. A process for purifying paliperidone, comprising:
a) providing a first solution of paliperidone in an organic solvent;
b) subjecting the first solution to silica gel treatment to provide a second solution; and
c) isolating highly pure paliperidone from the second solution obtained in step-(b), and optionally converting the highly pure paliperidone obtained into its pharmaceutically acceptable salts.

52. The process of claim 51, wherein the organic solvent used in step-(a) is selected from the group consisting of alcohols, amides, organosulfur solvents, and mixtures thereof.

53. The process of claim 52, wherein the organic solvent is selected from the group consisting of methanol, ethanol, n-propanol, isopropanol, isobutanol, n-butanol, tert-butanol, amyl alcohol, isoamyl alcohol, dimethylacetamide, dimethylformamide, dimethylsulfoxide, sulfolane, and mixtures thereof.

54. The process of claim 53, wherein the organic solvent is selected from the group consisting of methanol, isopropanol, dimethylformamide, sulfolane, and mixtures thereof.

55. The process of claim 51, wherein the subjecting to silica gel treatment in step-(b) is carried out by stirring the solution with silica gel at a temperature of below about 70° C. for at least 10 minutes.

56. The process of claim 51, wherein the isolation of highly pure paliperidone in step-(c) is carried out by forcible or spontaneous crystallization.

57. A pharmaceutical composition comprising highly pure paliperidone or a pharmaceutically acceptable salt thereof having a paliperidone keto impurity in an amount of less than about 0.2 wt % and one or more pharmaceutically acceptable excipients, wherein the paliperidone keto impurity is 3-[2-[4-(6-fluoro-1,2-benzisoxazol-3-yl)-1-piperidinyl]ethyl]-2-methyl-7,8-dihydro-6H-pyrido[1,2-a]pyrimidin-4,9-dione.

58. The pharmaceutical composition of claim 57, wherein the pharmaceutical composition is a solid dosage form, an oral suspension, a liquid, a powder, an elixir, an aerosol, syrups or an injectable solution.

59. The pharmaceutical composition of claim 57, wherein the highly pure paliperidone or a pharmaceutically acceptable salt thereof has a D₉₀ particle size of less than or equal to about 400 microns.

60. The pharmaceutical composition of claim 59, wherein the 90 volume-% of the particles (D₉₀) have a size of less than or equal to about 300 microns; less than or equal to about 100 microns; less than or equal to about 60 microns; or less than or equal to about 15 microns.

61. A method of treating a patient suffering from psychotic diseases, comprising administering a therapeutically effective amount of highly pure paliperidone or a pharmaceutically acceptable salt thereof having a paliperidone keto impurity in an amount of less than about 0.2 wt %, or a pharmaceutical composition that comprises a therapeutically effective amount of highly pure paliperidone or a pharmaceutically acceptable salt thereof having a paliperidone keto impurity in an amount of less than about 0.2 wt %, along with pharmaceutically acceptable excipients.
